(19) United States
(12) Patent Application Publication
(10) Pub. No.: US 2024/0065629 A1
(43) Pub. Date: Feb. 29, 2024

(54) A PRESSURE SENSING DEVICE AND RELATED METHODS

(71) Applicant: AGENCY FOR SCIENCE, TECHNOLOGY AND RESEARCH, Singapore (SG)

(72) Inventors: Maxim KIRYUKHIN, Singapore (SG); Su Hui LIM, Singapore (SG); Hooi Hong LAU, Singapore (SG); Zee UPTON, Singapore (SG); Melissa Laura FERNANDEZ, Singapore (SG)

(21) Appl. No.: 17/766,522

(22) PCT Filed: Oct. 9, 2020
(86) PCT No.: PCT/SG2020/050578

§ 371 Date: Apr. 4, 2022

(30) Foreign Application Priority Data
Oct. 11, 2019 (SG) 10201909539Q

(51) Int. Cl. A61B 5/00; A61F 13/08

(52) U.S. Cl. CPC A61B 5/6812; A61B 5/445; A61B 5/6828; A61F 13/08; A61B 2503/08; A61B 2562/0247

(57) ABSTRACT

There is provided a pressure sensing device and related methods, the pressure sensing device comprising, a plurality of chambers for storing one or more visual indicators, wherein the plurality of chambers are configured to release the one or more visual indicators when a pressure exerted on the plurality of chambers exceeds one or more pressure thresholds. The plurality of chambers can be microchambers. The device may be used in conjunction with a bandaging system to indicate the pressure applied onto a site of treatment.

### TECHNICAL FIELD

The present disclosure relates broadly to a pressure sensing device and related methods thereof.

### BACKGROUND

Limb ulcers e.g. chronic leg ulcers can cause pain, infection and reduction or loss of physical mobility, especially for elderly patients aged 65 years and above. In addition, chronic leg ulcers can be difficult to heal properly and are prone to recurrence. Consequently, chronic leg ulcers can have a significant impact on patients' quality of life.

Currently, compression therapy is accepted as the gold standard treatment of chronic leg ulcers, with a scarcity of viable alternative options. Compression therapy relies on the compression achieving a reduction in oedema and improved return of blood via the venous system. Compression may be achieved by the use of bandages e.g. elastic bandages to generate pressure on the affected limb where the ulcer(s) are located. The healing rates of compression therapy typically range from about 60% to 80%. The effectiveness of compression therapy depends on the amount of pressure applied to the affected limb. Ischemia and loss of limb may occur if the applied pressure is excessive (e.g. higher than mmHg). Ineffective compression therapy may occur if the applied pressure is inadequate (e.g. less than 20 mmHg).

Conventionally, compression therapy is administered by a specialised nurse who is required to undergo extensive skills training, supervision and a competency assessment to be able to practise safely and deliver optimal pressure to the affected limb. For example, a commercial Kikuhime pressure monitor may be used for training nurses to administer compression therapy. However, during actual administration of compression therapy, the pressure generated by the bandage on the limb is typically not measured. As such, the effectiveness of compression therapy can vary depending on the level of experience of the nurse administering the treatment and may not always reach the optimal pressure required for effective healing.

Thus, there is a need for a pressure sensing device and related methods that seek to address or at least ameliorate one or more of the above problems.

### SUMMARY

In one aspect, there is provided a pressure sensing device comprising, a plurality of chambers for storing one or more visual indicators, wherein the plurality of chambers are configured to release the one or more visual indicators when a pressure exerted on the plurality of chambers exceeds one or more pressure thresholds.

In one embodiment of the device as disclosed herein, the plurality of chambers comprise, a first subset of chambers configured to be collapsible to release a first visual indicator contained therein, when the pressure exerted on the plurality of chambers exceeds a first pressure threshold, a second subset of chambers configured to be collapsible to release a second visual indicator contained therein, when the pressure exerted on the plurality of chambers exceeds a second pressure threshold, wherein the second pressure threshold is higher than the first pressure threshold.

In one embodiment, the device further comprises, a first visual indicator stored within the first subset of chambers; and a second visual indicator stored within the second subset of chambers, wherein the release of only the first visual indicator indicates that the pressure exerted on the plurality of chambers is between the first and second pressure thresholds, and wherein the release of both the first and second visual indicators indicates that the pressure exerted on the plurality of chambers is above the second pressure threshold.

In one embodiment of the device as disclosed herein, the first pressure threshold is set at a level between 15 mmHg to 25 mmHg, and the second pressure threshold is set at a level between 25 mmHg to 45 mmHg.

In one embodiment of the device as disclosed herein, the second pressure threshold is at least 10 mmHg higher than the first pressure threshold.

In one embodiment of the device as disclosed herein, the pressure exerted on the plurality of chambers is a compressive pressure.

In one embodiment of the device as disclosed herein, the plurality of chambers comprise walls having a thickness of from 1 μm to 10 μm.

In one embodiment of the device as disclosed herein, the plurality of chambers is formed by a film comprising a single layer of polymer.

In one embodiment of the device as disclosed herein, the plurality of chambers is formed by a film comprising alternating complementary layers of polymers, wherein at least one alternating layer comprises polyanionic material selected from the group consisting of gum arabic, anionic polysaccharide, alginate, pectin, agar, carrageenan, polyacrylate, poly(4-styrenesulphonates), poly(vinylsulphonates), polyanetholesulphonates and combinations thereof; and wherein at least another alternating layer comprises polycationic material selected from the group consisting of gelatin, chitosan, whey proteins, albumin, beta-lactoglobulin, potato proteins, fava, legumin, soybean proteins, natural or synthetic polysaccharides, chitosan, poly(allylammonium), poly(diallyldimethylammonium), poly(ethyleneimine) (PEI), polyl-lysine (PLL), poly(histidine), heparin, heparan sulfate, chondroitin sulfate, dextran sulfate, oxidized cellulose, polyaspartic acid, polyglutamic acid and combinations thereof.

In one embodiment of the device as disclosed herein, the film comprises at least 10 layers of polymers.

In one embodiment of the device as disclosed herein, the plurality of chambers is sealed by a support layer to prevent and/or minimise leakage of the one or more visual indicators from said chambers.

In one aspect, there is provided a method of making a pressure sensing device, the method comprising, forming a film comprising a plurality of chambers for storing one or more visual indicators, wherein the plurality of chambers are configured to release the one or more visual indicators when a pressure exerted on the plurality of chambers exceeds one or more pressure thresholds.

In one embodiment, the method further comprises, prior to forming the film, fabricating a pattern template comprising a plurality of wells, wherein said plurality of wells is a template for the plurality of chambers.

In one embodiment of the method as disclosed herein, the step of forming the film comprises depositing a polymeric material on the pattern template to form a single layer of polymer, or depositing two or more polymeric materials in an alternating manner to form alternating layers of complementary polymers.

In one embodiment of the method as disclosed herein, the step of forming the film comprises forming a plurality of chambers with walls having a thickness of from 1 μm to 10 μm.

In one embodiment, the method further comprises applying a support layer to the film to prevent and/or minimise leakage of the one or more visual indicators from the plurality of chambers.

In one embodiment, the method further comprises loading one or more visual indicators into the plurality of chambers using an ink-jet printer or a solvent exchange technique.

In one aspect, there is provided a bandaging system comprising, one or more pressure sensing devices as disclosed herein.

In one embodiment, the bandaging system further comprises a primary bandaging layer for covering a site of interest, wherein the one or more pressure sensing devices are arranged to be positioned on the primary bandaging layer.

In one embodiment, the bandaging system further comprises one or more secondary bandaging layers for exerting pressure on the site of interest, wherein the one or more secondary bandaging layers are arranged to cover the one or more pressure sensing devices positioned on the primary bandaging layer.

In one embodiment of the bandaging system as disclosed herein, the one or more visual indicators are arranged to penetrate the one or more secondary bandaging layers to form one or more visually observable markings thereon when they are released from the plurality of chambers.

In one aspect, there is provided a method of detecting applied pressure, the method comprising, positioning one or more pressure sensing devices as disclosed herein on a site of interest, applying pressure on the site of interest, and detecting one or more visually observable signals formed by the one or more visual indicators released from the plurality of chambers.

In one embodiment of the method as disclosed herein, the step of applying pressure comprises, increasing pressure on the site of interest until release of a first visual indicator is detected but before release of a second visual indicator is detected, indicating that the pressure applied on the site of interest exceeds a first pressure threshold but not a second pressure threshold.

In one embodiment, the method further comprises, prior to positioning the one or more pressure sensing devices, covering the site of interest with a primary bandaging layer.

In one embodiment, the method further comprises applying one or more secondary bandaging layers for exerting pressure on the site of interest, wherein the one or more secondary bandaging layers are arranged to cover the one or more pressure sensing devices positioned on the primary bandaging layer.

In one embodiment of the method as disclosed herein, detection of the release of one or more visual indicators is through one or more visually observable signals formed on the one or more secondary bandaging layers.

### Definitions

The term “visual indicator” as used herein broadly refers to a substance which is capable of producing a visually observable signal. The visually observable signal may be a signal which is observable using the naked eye or a signal which is observable with the aid of an instrument e.g. an ultraviolet lamp. For example, the visual indicator may be a coloured dye, a fluorescent dye, or a combination thereof.

The term “ulcer” as used herein includes an open sore or lesion of the skin that involves the sloughing off of inflamed and necrotized tissue and includes, but is not limited to, callous ulcers, chronic leg ulcers, decubitus, and any type of secondary lesion that is a breach of the cornified and the epidermal layer of the skin. In one example, the one or more ulcers comprise chronic leg ulcers of venous and/or mixed etiology.

The terms “site of interest”, “target site” and “specific site” as used herein broadly refer to a surface at a particular region of a subject. For example, the subject may be a mammal e.g. human. The surface may refer to a skin surface of the human subject. The region may refer to a limb e.g. leg of the human subject. For example, the site of interest may refer to a skin surface of a subject where one or more ulcers are located.

The term “substrate” as used herein is to be interpreted broadly to refer to any supporting structure.

The term “layer” when used to describe a first material is to be interpreted broadly to refer to a first depth of the first material that is distinguishable from a second depth of a second material. The first material of the layer may be present as a continuous film, as discontinuous structures or as a mixture of both. The layer may also be of a substantially uniform depth throughout or varying depths. Accordingly, when the layer is formed by individual structures, the dimensions of each of individual structure may be different. The first material and the second material may be same or different and the first depth and second depth may be same or different.

The term “complementary” when used to describe a relationship between adjacent layers of materials (e.g. a first layer and a second layer of polymers) is to be interpreted broadly to refer to characteristics of the materials which enable the adjacent layers of materials to be coupled together. Coupling of complementary layers of materials may be achieved via various types of interactions between the layers, which include but are not limited to hydrogen bonding, hydrophobic interactions, charge interactions etc. For example, the first and second layers of polymers may have opposite charges which enable coupling of the two layers of polymers.

The term “continuous” when used to describe a film or a layer is to be interpreted broadly to refer to a film or a layer that is substantially without significant breaks in continuity across the film or layer. In this regard, a continuous film or a continuous layer is also intended to include a film or a layer that may have trivial gaps or holes or voids that may not appreciably affect the desired properties of the film or the layer.

The term “micro” as used herein is to be interpreted broadly to include dimensions from about 1 micron to about 1000 microns.

The term “nano” as used herein is to be interpreted broadly to include dimensions less than about 1000 nm.

The terms “coupled” or “connected” as used in this description are intended to cover both directly connected or connected through one or more intermediate means, unless otherwise stated.

The term “associated with”, used herein when referring to two elements refers to a broad relationship between the two elements. The relationship includes, but is not limited to a physical, a chemical or a biological relationship. For example, when element A is associated with element B, elements A and B may be directly or indirectly attached to each other or element A may contain element B or vice versa.

The term “adjacent” used herein when referring to two elements refers to one element being in close proximity to another element and may be but is not limited to the elements contacting each other or may further include the elements being separated by one or more further elements disposed therebetween.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. It is to be appreciated that the individual numerical values within the range also include integers, fractions and decimals. Furthermore, whenever a range has been described, it is also intended that the range covers and teaches values of up to 2 additional decimal places or significant figures (where appropriate) from the shown numerical end points. For example, a description of a range of 1% to 5% is intended to have specifically disclosed the ranges 1.00% to 5.00% and also 1.0% to 5.0% and all their intermediate values (such as 1.01%, 1.02% . . . 4.98%, 4.99%, 5.00% and 1.1%, 1.2% . . . 4.8%, 4.9%, 5.0% etc.) spanning the ranges. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

### DESCRIPTION OF EMBODIMENTS

Non-limiting embodiments of a pressure sensing device and related methods are disclosed hereinafter.

In various embodiments, there is provided a pressure sensing device comprising a plurality of chambers for holding/containing/storing one or more visual indicators, wherein the plurality of chambers are configured to release the one or more visual indicators when a pressure exerted on the plurality of chambers exceeds one or more pressure thresholds.

In various embodiments, the pressure sensing device is a pressure sensor, a pressure monitor and/or pressure indicator. The pressure sensing device may be used in various applications where the sensing of, or an indication of one or more pressure levels is required. For example, the device may be used in conjunction with a compression bandaging system to indicate the pressure applied onto a site of treatment. In another example, the device may be used in conjunction with pressure garments to provide an indication of the actual pressure being delivered to burns patients wearing pressure garments to reduce scarring. In another example, the device may be used in conjunction with compression stockings to provide an indication of the actual pressure being delivered to patients wearing compression stockings after surgical procedures. In yet another example, the device may be used in conjunction with compression socks for flying to check that the compression socks for flying are exerting the appropriate levels of compression to e.g. increase the blood flow in the user's lower legs, thereby reducing the risk of blood clots and swollen feet from flying. Accordingly, the pressure sensing device may be an integrated device that is integrated in textile materials suitable for use on a subject e.g. patients. Alternatively, the device may be a standalone device that can be applied on a target site or used in conjunction with a separate textile material.

In various embodiments, the pressure sensing device may be used to improve the efficacy of compression/compressive therapy at a site of treatment comprising one or more ulcers. In various embodiments, the pressure sensing device may be used in conjunction with, or integrated into compression bandages to effectively indicate one or more levels of compressive pressures. In various embodiments, the pressure sensing device may be used in detecting pressure of compression bandages for the treatment of chronic leg ulcers via the use of visual indicators e.g. coloured dyes to indicate the presence of one or more pressure threshold levels. In various embodiments, the one or more pressure thresholds may comprise a minimum pressure threshold for informing a user that the pressure applied to the site of treatment is effective for compressive therapy, and a maximum pressure threshold for informing the user that the pressure applied to the site of treatment is unsafe. For example, the pressure sensing device may be used to indicate two levels of compressive pressures at more than 20 mmHg and more than 40 mmHg. The use of the pressure sensor may advantageously improve efficacy and efficiency of compressive therapy using compression bandages for chronic leg ulcers of mixed etiology.

In various embodiments, the plurality of chambers is configured to collapse/rupture/break when the pressure exerted on the plurality of chambers exceeds the one or more pressure thresholds. In various embodiments, the plurality of chambers are configured to deform substantially uniformly under pressure. In various embodiments, deformation of the plurality of chambers in response to pressure exerted thereon results in the collapse/rupture/breakage of the plurality of chambers, thus releasing the one or more visual indicators. In various embodiments, the pressure sensing device is a disposable sensor. In various embodiments, the pressure sensing device is configured for one-time/single use e.g. not reusable after the chambers are ruptured.

In various embodiments, the pressure exerted on the plurality of chambers is a compressive pressure. In various embodiments, the release of the one or more visual indicators is due to the compressive pressure exceeding one or more pressure thresholds. In various embodiments, the release of the one or more visual indicators is not due to a change in conditions such as pH, temperature, ionic strength, magnetic field, ultrasound, light, presence of concentration of a solvent, concentration of a trigger compound, and a combination thereof. For example, the release of the one or more visual indicators is not due to a change in osmotic pressure inside and outside the plurality of chambers. In another example, the release of the one or more visual indicators is not due to a change in chemical concentration of the one or more visual indicators within the plurality of chambers.

In various embodiments, the pressure sensing device is substantially devoid of pneumatic, electrical, optical and electronic components. In various embodiments, electrical or electronic components include but are not limited to piezoelectric/capacitive/resistive sensors and transducers, force-sensitive resistor, strain gauge and the like. In various embodiments, optical components may include but are not limited to electromagnetic wave emitter or receiver e.g. laser. In various embodiments, pneumatic components include but are not limited to manometer, air balloon and the like. In various embodiments, sensing/detection of pressure threshold levels is not based on electromechanical effects such as piezoelectric effect.

In various embodiments, the plurality of chambers comprise a first subset of chambers configured to be collapsible to release a first visual indicator contained therein, when the pressure exerted on the plurality of chambers exceeds a first pressure threshold. In various embodiments, the pressure sensing device further comprises the first visual indicator stored within the first subset of chambers

In various embodiments, the first pressure threshold is no less than about mmHg, no less than about 20 mmHg, no less than about 25 mmHg, no less than about 30 mmHg, no less than about 35 mmHg, no less than about 40 mmHg, or no less than about 45 mmHg. In various embodiments, the first subset of chambers may be designed such that the first pressure threshold is set at a level between about 15 mmHg to about 25 mmHg. In one example embodiment, the first pressure threshold is about 20 mmHg. The first pressure threshold may be a minimum amount of pressure required to be exerted for an application. For example, the first pressure threshold may be a minimum pressure threshold for informing a user that the pressure applied to a site of treatment is effective for compression therapy.

In various embodiments, the plurality of chambers further comprise a second subset of chambers configured to be collapsible to release a second visual indicator contained therein, when the pressure exerted on the plurality of chambers exceeds a second pressure threshold. In various embodiments, the pressure sensing device further comprises the second visual indicator stored within the second subset of chambers

In various embodiments, the second pressure threshold is no less than about 25 mmHg, no less than about 30 mmHg, no less than about 35 mmHg, no less than about 40 mmHg, no less than about 45 mmHg, no less than about 50 mmHg, no less than about 55 mmHg, or no less than about 60 mmHg. In various embodiments, the second subset of chambers may be designed such that the second pressure threshold is set at a level between about 25 mmHg to about 45 mmHg. In one example embodiment, the second pressure threshold is about 40 mmHg. The second pressure threshold may be a maximum amount of pressure which can be exerted for an application. For example, the second pressure threshold may be a maximum pressure threshold for informing the user that the pressure applied to a site of treatment has reached an unsafe level.

In various embodiments, the second pressure threshold is higher than the first pressure threshold. In various embodiments, the second pressure threshold may be at least 5 mmHg, at least 10 mmHg, at least 15 mmHg, at least 20 mmHg, at least 25 mmHg, at least 30 mmHg, at least 35 mmHg, at least 40 mmHg, or at least 45 mmHg higher than the first pressure threshold. For example, the second pressure threshold may be at least about 2 times higher than the first threshold.

In various embodiments, the second subset of chambers remain substantially intact when the pressure exerted on the plurality of chambers is between first and second pressure thresholds. That is, when the pressure exerted on the plurality of chambers is above the first pressure threshold but below the second pressure threshold, the first subset of chambers collapse/rupture/break and release the first visual indicator contained therein while the second subset of chambers remain substantially intact (i.e. not collapsed/ruptured/broken). When the pressure exerted on the plurality of chambers is above the second pressure threshold, the second subset of chambers collapse/rupture/break and release the second visual indicator contained therein.

In various embodiments, the release of only the first visual indicator indicates that the pressure exerted on the plurality of chambers is between the first and second pressure thresholds. In various embodiments, the release of both the first and second visual indicators indicates that the pressure exerted on the plurality of chambers is above the second pressure threshold.

In various embodiments, the first visual indicator is configured to produce a first visual signal indicating that the first pressure threshold is exceeded. In various embodiments, the second visual indicator is configured to produce a second visual signal indicating that the second pressure threshold is exceeded. In various embodiments, the first visual signal is visually distinct from the second visual signal such that a user is capable of distinguishing based on the visual signals whether the first or second pressure threshold is exceeded. For example, the first and second visual signals may be distinguished from each other by different colours, number of marks/stains formed by the visual indicators, position of the marks/stains, size of the marks/stains, pattern of the marks/stains and the like. In some embodiments, the first and second visual indicators for producing the first and second visual signals may be different in colour. In some embodiments, the first and second visual indicators may be of the same colour, but the chambers in the first and second subsets of chambers may be arranged in different patterns such that the first and second visual signals are of the same colour but of different patterns.

It will be appreciated that the above description in relation to the first and second subsets of chambers is for illustrative purposes and that the pressure sensing device may comprise further subsets of chambers configured to be collapsible when the pressure exerted on the plurality of chambers exceeds a further pressure threshold. For example, there may be a third subset of chambers configured to be collapsible when the pressure exerted on the plurality of chambers exceeds a third pressure threshold.

In various embodiments, the pressure exerted on the plurality of chambers is a compressive pressure. Accordingly, the first and second pressure thresholds may refer to compressive pressure thresholds.

In various embodiments, in an application for a compression bandaging system, the inventors have recognised that the optimal and therapeutic levels of compression differ from one country to another. The inventors have also recognised that the sub-bandage pressure produced by a compression bandaging system may vary based on the physical activity of the patient. In various embodiments, patients diagnosed with ulcers of mixed etiology may require reduced compression of about 15-25 mmHg. In various embodiments, for patients diagnosed with ulcers of mixed etiology, compression higher than 15-25 mmHg may cause occlusion and further damage to the limb. In various embodiments, compression bandaging of about 35-45 mmHg has been shown to be safe and effective. In various embodiments, the first pressure threshold may be set to represent the minimum pressure level required for compression therapy to be effective. In various embodiments, the second pressure threshold may be set to represent the maximum pressure level which can be applied to the limb, beyond which side effects such as occlusion and damage to the limb may occur.

In various embodiments, the pressure sensing device may be configured to have a sheet-like structure. In various examples, the pressure sensing device may be configured in different shapes and sizes to suit specific requirements of an application. For example, such requirements may include but are not limited to the topology and surface area of a site where the device is applied thereto. For example, the pressure sensing device may be configured into various geometric shapes such as a strip, square, circle and the like.

In various embodiments, the pressure sensing device is configured to be substantially flat, flexible and/or pliable. In an application for compression bandaging, the pressure sensing device may be dimensioned to be relatively thin and substantially flexible such that a patient may not perceive/sense/feel its presence under the bandaging. Advantageously, the pressure sensing device may not affect uniformity of delivered compression by the bandaging.

In various embodiments, the plurality of chambers having different subsets of chambers are arranged such that merging, smudging and/or mixing of visual indicators from different subsets of chambers with different pressure thresholds are avoided. For example, the first subset of chambers may be comprised on a first body of the device e.g. strip and the second subset of chambers may be comprised on a second body of the device e.g. strip. Each strip comprising one subset of chambers is configured to release the visual indicator contained therein at a pressure threshold which may be the same or different pressure threshold from another strip.

In various embodiments, different subsets of chambers may be comprised on the same body of the device e.g. strip. For example, the first and second subsets of chambers may be formed on the same strip (e.g. in an interspersed or segregated manner) but sufficiently spaced apart or separated from each other by a separator to prevent merging, smudging and/or mixing of visual indicators. For the case where different subsets of chambers are comprised on the same body, the first and second visual indicators contained within the first and second subsets of chambers may be distinguished from each other by e.g. using different colours to enable ease of differentiation.

In various embodiments, the plurality of chambers having different subsets of chambers may be arranged to facilitate merging/mixing/reaction of visual indicators from different subsets of chambers with different pressure thresholds, when the visual indicators are released from the respective subsets of chambers. For example, the second visual indicator and/or a substance contained within the second subset of chambers may be configured to merge/mix/react with the first visual indicator and/or another substance contained within the first subset of chambers, when released from the respective subsets of chambers. The merging/mixing/reaction may result in the formation of a visual signal which is visually distinct (e.g. a change to a different colour) from the first visual signal produced by the first visual indicator.

It will be appreciated that other configurations or arrangements of the different subsets of chambers (e.g. the first and second subsets of chambers) may also be possible as long as the release of visual indicators results in different visually distinct signals at different pressure thresholds.

In various embodiments, the pressure sensing device comprises a film where the plurality of chambers is formed thereon. Each of the plurality of chambers may form a protrusion on the surface of the film. The plurality of chambers may be arranged to form an array e.g. two-dimensional array. The term “array” as used herein broadly refers to an ordered arrangement of features on a surface. In various embodiments, each of the plurality of chambers is formed from the same composition as the film. In various embodiments, composition includes but is not limited to structural composition, chemical composition and the like. In various embodiments, the one or more pressure thresholds are a function of the thickness of the chamber, material used for the chamber, and/or geometry of the chamber. Accordingly, in various embodiments, the second subset of chambers may differ from the first subset of chambers by thickness or not by thickness but by the material used.

In various embodiments, the film is a single-layered film. In various embodiments, the single-layered film may be formed from a single polymeric material, or a composite/blend comprising two or more polymeric materials. Polymeric materials which are suitable for forming the single-layered film may include but are not limited to polylactic acid, poly(butylene succinate), polycaprolactone (PCL), polyhydroxy alkanoates (PHAs), polyhydroxybutyrates (PHBs), polyhydroxyvalerates (PHVs), thermoplastic starch (TPS) or aliphatic and aliphatic-aromatic polyesters and combinations thereof.

In various embodiments, the film is a multilayered film comprising a plurality of sub-layers. In various embodiments, the plurality of sub-layers comprises compositions different from each other e.g. in terms of materials, chemical or physical properties, dimensions such as thicknesses etc. The plurality of sub-layers may be coupled via various types of interactions between the layers, which include but are not limited to hydrogen bonding, hydrophobic interactions, charge interactions etc. In one example, the film forming the plurality of chambers may be made from protein-polyphenol-clay layered composite material.

In various embodiments, the film comprises components which include but are not limited to organic substances, inorganic substances, polymers, and non-polymers. The film may include one or more components selected from the group consisting of organic substances, inorganic substances, polymers, and non-polymers. For example, the components may include but are not limited to polyelectrolytes, polymeric species, inorganic substances, proteins, and the like.

In various embodiments, the film comprises alternating layers of polymers. In various embodiments, the film comprises alternating complementary layers of polymers. In various embodiments, the alternating layers of polymers may have opposite charges. The film may comprise alternating sub-layers of a first polymer and a second polymer. In various embodiments, each complementary pair of layers or each pair of layers formed from opposite charges may be termed as a bilayer. It will also be appreciated that in other embodiments, different combinations or arrangements of the sub-layers in the film may be contemplated. The first and second polymer sub-layers may include but are not limited to cationic polymer, an anionic polymer, a nonionic polymer, an amphoteric polymer and combinations thereof. The first and second polymer sub-layers may be selected from the group consisting of a cationic polymer, an anionic polymer, a nonionic polymer, an amphoteric polymer and combinations thereof.

In various embodiments, the first and second polymer sub-layers are formed from polymers which include but are not limited to poly(sodium 4-styrene sulfonate) (PSS), poly(allylamine hydrochloride) (PAH), poly-lysine (PLL), poly-arginine (PARG), poly(ethylene imine) (PEI), poly(histidine), poly-diallyldimethylammonium chloride, poly(acrylic acid) (PAA), alginate, hyaluronic acid, heparin, heparan sulfate, chondroitin sulfate, dextran sulfate, poly(methacrylic acid), oxidized cellulose, carboxymethyl cellulose, polyaspartic acid, polyglutamic acid and natural or synthetic polysaccharides such as chitosan. For example, the first polymer sub-layer may comprise PSS and the second polymer sub-layer may comprise PAH. The exterior-most layer of the film may comprise PSS.

In various embodiments, the polymer sub-layer may be formed from anionic polymers selected from the group consisting of gum arabic, anionic polysaccharide, alginate, pectin, agar, carrageenan, polyacrylate, poly(4-styrenesulphonates), poly(vinylsulphonates), polyanetholesulphonates and combinations thereof.

In various embodiments, the polymer sub-layer may be formed from cationic polymers selected from the group consisting of gelatin (such as gelatin A or gelatin B), chitosan, whey proteins, albumin, beta-lactoglobulin, potato proteins, fava, legumin, soybean proteins, natural or synthetic polysaccharides such as chitosan, poly(allylammonium), poly(diallyldimethylammonium), poly(ethyleneimine) (PEI), polyl-lysine (PLL), poly(histidine), hyaluronic acid, heparin, heparan sulfate, chondroitin sulfate, dextran sulfate, poly(methacrylates), oxidized cellulose, polyaspartic acid, polyglutamic acid and combinations thereof.

In various embodiments, each of the plurality of chambers comprises one or more walls defining an internal space for containing/holding/storing the visual indicator. In various embodiments, each of the plurality of chambers defines a geometric shape. In various examples, a geometric shape includes but is not limited to a sphere, hemisphere, ellipsoid, cylinder, cube, cuboid, cone, truncated cone, pyramid (e.g. triangular pyramid, square pyramid, polygonal pyramid, right pyramid, oblique pyramid etc.), truncated pyramid and the like. When viewed from above the film, each chamber may have a cross-section in the shape of a circle, ellipse, oval, triangle, parallelogram, trapezoid, rectangle, square, polygon and the like. For example, each of the plurality of chambers may comprise a substantially square base and four substantially rectangular side walls.

In various embodiments, each chamber may be configured to rupture/collapse at a predetermined pressure threshold by customising one or more parameters of the chamber. The one or more parameters include but are not limited to the geometry of the chamber, chemical or material composition of the chamber, thickness of the chamber wall (e.g. multilayer film used to form the chamber wall) and the like. Customisation of the one or more parameters may allow the chamber to collapse under a desired compression pressure, thereby releasing the visual indicator e.g. coloured payload.

In various embodiments, the plurality of chambers are microchambers, i.e. micron-sized chambers. In various embodiments, each of the plurality of chambers comprises dimensions which are in the micrometer order of magnitude. In various examples, dimensions include but are not limited to length, width, thickness, diameter and the like.

In various embodiments, the micrometer order of magnitude is defined not more than about 1000 μm, not more than about 950 μm, not more than about 900 μm, not more than about 850 μm, not more than about 800 μm, not more than about 750 μm, not more than about 700 μm, not more than about 650 μm, not more than about 600 μm, not more than about 550 μm, not more than about 500 μm, not more than about 450 μm, not more than about 400 μm, not more than about 350 μm, not more than about 300 μm, not more than about 250 μm, not more than about 200 μm, not more than about 150 μm, not more than about 100 μm, not more than about 50 μm, not more than about 40 μm, not more than about 30 μm, not more than about 20 μm, or not more than about 10 μm.

In various embodiments, depending on the geometry of each chamber, the longest dimension (i.e. one of height, length, width, or diameter) of each chamber may be from about 50 μm to about 1000 μm, from about 100 μm to about 950 μm, from about 150 μm to about 900 μm, from about 200 μm to about 850 μm, from about 250 μm to about 800 μm, from about 300 μm to about 750 μm, from about 350 μm to about 700 μm, from about 400 μm to about 650 μm, from about 450 μm to about 600 μm, or from about 500 μm to about 550 μm. In one example, for a chamber comprising a substantially square base and four substantially rectangular side walls, the square base measures about 150 μm (length) by about 150 μm (width) and each rectangular side wall measures about 150 μm (length) by about 100 μm (height).

In various embodiments, the film comprising the plurality of chambers has a minimal thickness in order to maintain the structure and shape of the chamber up to a desired pressure threshold, and to prevent collapse of the chambers before the desired pressure threshold is reached or exceeded. In various embodiments, the film and each of the plurality of chambers comprise walls having a thickness from about 0.5 μm to about 10 μm, from about 1 μm to about 9.5 μm, from about 1.5 μm to about 9 μm, from about 2 μm to about 8.5 μm, from about 2.5 μm to about 8 μm, from about 3 μm to about 7.5 μm, from about 3.5 μm to about 7 μm, from about 4 μm to about 6.5 μm, from about 4.5 μm to about 6 μm, or from about 5 μm to about 5.5 μm. In one embodiment, the film and each of the plurality of chambers comprise walls having a thickness from about 1.8 μm to about 1.9 μm.

In various embodiments, where the film is formed from alternating layers of polymers having opposite charges, the number of polymer layers used to formed the film may depend on the size and shape of the chamber and the Young's modulus of the film. In general, the number of polymer layers required for forming a chamber increases with the size of the chamber. The higher the Young's modulus of the film material, the thinner the film can be. In various embodiments, the Young's modulus of film may be in the range from about 0.2 GPa to about 60 GPa, from about 0.4 GPa to about 58 GPa, from about 0.6 GPa to about 56 GPa, from about 0.8 GPa to about 54 GPa, from about 1 GPa to about 52 GPa, from about 2 GPa to about 50 GPa, from about 4 GPa to about 48 GPa, from about 6 GPa to about 46 GPa, from about 8 GPa to about 44 GPa, from about 10 GPa to about 42 GPa, from about 12 GPa to about 40 GPa, from about 14 GPa to about 38 GPa, from about 16 GPa to about 36 GPa, from about 18 GPa to about 34 GPa, from about 20 GPa to about 32 GPa, from about 22 GPa to about 30 GPa, from about 24 GPa to about 28 GPa, or from about 25 GPa to about 26 GPa. In various embodiments, the tensile strength of film may be in the range from about 10 MPa to about 320 MPa, from about 20 MPa to about 300 MPa, from about 40 MPa to about 280 MPa, from about 60 MPa to about 260 MPa, from about 80 MPa to about 240 MPa, from about 100 MPa to about 220 MPa, from about 120 MPa to about 200 MPa, from about 140 MPa to about 180 MPa, or from about 150 MPa to about 160 MPa. In some embodiments, the film formed from alternating complementary layers of polymers may have a Young's modulus from about 4 GPa to about 6 GPa and/or a tensile strength from about 20 MPa to about 40 MPa. In one embodiment, the film may be formed from poly(ethyleneimine)-montmorillonite multilayer composites, having a Young's modulus of about 60 GPa and/or a tensile strength of about 320 MPa. In one embodiment, the film may be wet poly(styrene sulfonate)-poly(allylamine) multilayer films, having a Young's modulus of about 0.2 GPa and/or a tensile strength of about 10 MPa. In one embodiment where the first layer is made up of alternating layers of PAH-PSS, the Young's modulus of film is in the range of about 4 to about 6 GPa and the tensile strength of film is in the range of about 20 to about 40 MPa. The upper limit to the number of polymer layers may be defined by a ratio between the film thickness and the dimension (e.g. diameter, length or width) of the chamber, such that a space is still provided within the chamber for storing the visual indicator.

In various embodiments, where the film is formed from alternating layers of polymers having opposite charges, the film may comprise at least 10 layers of polymers. In various embodiments, the film may comprise from about 10 to about 200 layers of polymers (or from about 5 to about 100 bilayers), from about 20 to about 190 layers of polymers, from about 30 to about 180 layers of polymers, from about 40 to about 170 layers of polymers, from about 50 to about 160 layers of polymers, from about 60 to about 150 layers of polymers, from about 70 to about 140 layers of polymers, from about 80 to about 130 layers of polymers, from about 90 to about 120 layers of polymers, or from about 100 to about 110 layers of polymers. In one embodiment, the film may comprise from about 80 to about 160 layers of polymers (or from about 40 to about 80 bilayers).

In various embodiments, the plurality of chambers is sealed by a support layer to prevent and/or minimise leakage of the one or more visual indicators from said chambers. In various embodiments, the pressure sensing device further comprises the support layer disposed adjacent to the film. In various embodiments, the support layer is configured to seal/cover the plurality of chambers to prevent and/or minimise leakage of the one or more visual indicators. In various embodiments, the support layer substantially/completely seals the chambers. In various embodiments, the one or more visual indicators may be loaded into the plurality of chambers using a suitable technique depending on the characteristics or properties of the one or more visual indicators and the plurality of chambers. In various embodiments, an inkjet printer/plotter is used for loading the one or more visual indicators into the plurality of chambers. In various embodiments, a solvent-exchange method is used to load the one or more visual indicators into the plurality of chambers. In various embodiments, the one or more visual indicators are loaded into the plurality of chambers before the support layer substantially/completely seals the plurality of chambers. In various embodiments, the one or more visual indicators are loaded into the plurality of chambers after the support layer substantially/completely seals the plurality of chambers. In various embodiments, the support layer is substantially or completely impermeable to the visual indicator.

In various embodiments, the support layer is a polymeric support, i.e. made from one or more polymers. In various embodiments, the support layer comprises a polymer composition that is distinct from the film e.g. multilayered film where the plurality of chambers is disposed. The one or more polymers may include but are not limited to polyethylene, high-density polyethylene, low-density polyethylene, linear low-density polyethylene, polypropylene, high crystalline polypropylene, polyethylene-propylene copolymer, polyethylene-butylene copolymer, polyethylene-hexene copolymer, polyethylene-octene copolymer, polystyrene-butylene-styrene copolymer, polystyrene-ethylene-butylene-styrene copolymer, polystyrene, polyphenylene oxide, polysulfone, polycarbonate, polyester, polyamide, polyurethane, polyacrylate, polyvinylchloride, polyethylene terephthalate, polyvinylidene chloride, polyvinylidene fluoride, polysiloxane, polyolefin ionomer, polymethyl pentene, hydrogenated oligocyclopentadiene (HOCP), and a copolymer thereof or a derivative thereof. In one example, the support layer comprises polyethylene.

In various embodiments, the pressure sensing device further comprises an adhesive layer disposed between the support layer and the film. In various embodiments, the support layer is adhered to the film via the adhesive layer. In various embodiments, the adhesive layer is multilayered. In various embodiments, the adhesive layer has a thickness of from about 0.3 μm to about 10 μm, from about 0.5 μm to about 9 μm, from about 1 μm to about 8 μm, from about 2 μm to about 7 μm, from about 3 μm to about 6 μm, or from about 4 μm to about 5 μm.

In various embodiments, the one or more visual indicators comprise one or more dyes capable of penetrating one or more layers of materials used to make bandage. In various embodiments, the one or more dyes are water-soluble or oil-soluble dyes, and/or are natural or synthetic dyes.

In various embodiments, the one or more dyes corresponding to the one or more pressure thresholds are represented by the same colour of dye. That is, instead of using different colours to represent the number of pressure thresholds exceeded, the number of marks/stains formed by the dye (which is of the same colour) may be used to indicate the number of pressure thresholds exceeded. For example, when none of the pressure threshold(s) is exceeded, there is no mark/stain formed by the dye. When a first (lower) pressure threshold is exceeded, there is one mark formed. When a second (higher) pressure threshold is exceeded, there are two marks formed.

In various embodiments, each of the one or more dyes corresponds to one of the one or more pressure thresholds, such that each pressure threshold is represented by a unique identifier e.g. unique colour of dye spots, unique number of dye spots, unique position of dye spots, unique size of dye spots, unique pattern of dye spots, unique combination of dye spots etc.

In various embodiments, the one or more visual indicators are in the liquid state. In various embodiments, the one or more visual indicators are capable of wetting or penetrating one or more layers of material used to make bandage. In various examples, material for making bandage include but is not limited to foamed, woven or nonwoven material of natural or synthetic fibers such as rayon, polyester, polyurethane, polyolefin, cellulose, cellulose derivatives, cotton, orlon and the like.

In various embodiments, the one or more visual indicators are configured to form one or more marks/stains which are observable with the naked eye. In various embodiments, the formation of one or more marks/stains on the material for making bandage is a result of the one or more visual indicators penetrating and wetting said material. In various embodiments, the formation of one or more marks/stains on the material for making bandage is not a result of a change in the property of the material for making bandage e.g. mechanical property such as stress, strain, and the like.

In various embodiments, the one or more visual indicators are safe for human contact. Safe for human contact as used herein means that the visual indicators are non-hazardous and will not cause adverse reaction(s) to a human subject in the event of physical contact with the skin surface e.g. healthy skin or skin with an open wound. In various embodiments, the one or more visual indicators may comprise a fluorescent dye (3,4,9,10-tetra-(hectoxy-carbonyl)-perylene) dissolved in oil e.g. an edible oil such as a sunflower oil.

In various embodiments, the pressure sensing device is configured to operate in a substantially dry e.g. non-aqueous environment. In various embodiments, operating in a dry environment allows the marks/stains formed by the one or more visual indicators to be easily observed.

In various embodiments, the pressure sensing device e.g. pressure sensor may comprise a first film layer containing an array of chambers, each chamber filled with a dye solution and sealed by a second support/film layer. In various embodiments, the pressure sensor is based on the release of encapsulated dye upon collapse of the chambers e.g. microchambers. In various embodiments, the pressure sensor relies on release of dye upon pressure-induced collapse of microchambers and do not require any additional component e.g. electrical or electronic components. In various embodiments, pressure sensor further comprises a bandage or is configured to be used in conjunction with a compressive bandage. In various embodiments, the pressure sensor is a standalone device. In various embodiments, the pressure sensor is not integrated with a bandage or material used to make bandage.

In various embodiments, there is provided a method/process of making a pressure sensing device, the method comprising, forming a film comprising a plurality of chambers for containing one or more visual indicators, wherein the plurality of chambers are configured to release the one or more visual indicators when a pressure exerted on the plurality of chambers exceeds one or more pressure thresholds.

In various embodiments, the method further comprises, prior to forming the plurality of chambers, fabricating a pattern template comprising a plurality of wells, wherein said plurality of wells is a template for the plurality of chambers. In various embodiments, the pattern template serves as a guide/template for formation/deposition of the film comprising the plurality of chambers. For example, the pattern template may be a negative image of the plurality of chambers. The pattern template may be a sacrificial template which can be destroyed or removed during the process of making the pressure sensing device such that the pattern template does not form part of the plurality of chambers.

In various embodiments, fabricating the pattern template comprises forming/imprinting/embossing/casting the plurality of wells on one surface of a substrate. In various embodiments, imprinting the plurality of wells comprises applying the substrate onto a mold/stamp. In various embodiments, the method comprises making the mold/stamp prior to fabricating the pattern template. The step of making the mold/stamp may be performed by techniques e.g. lithographic techniques such as electron beam lithography, photolithography, micromachining and the like.

In various embodiments, the mold may comprise a defined imprint forming surface and may be applied onto the substrate at a defined temperature and pressure for a defined period of time. The temperature, pressure and time duration to be applied for imprinting may depend on the polymer substrate used and the imprints to be formed on the surface of the polymer substrate. The mold may be made of any suitable material that is chemically inert and may be harder than the softened substrate when used at the defined temperature. The mold may have a low surface energy so that any demolding can be carried out without destroying the imprinted structure. The mold may be selected from the group consisting of silicon, metal, glass, quartz, ceramic and combinations thereof.

In various embodiments, the pattern template may be formed using any suitable material which is capable of being imprinted by the mold/stamp. For example, the pattern template may be formed using a polymeric material. In various examples, the polymeric material comprises thermoplastic polymers which include but are not limited to acrylates, phthalimides, acrylonitriles, cellulosics, styrenes, alkyls, alkyls methacrylates, alkenes, halogenated alkenes, amides, imides, aryl ether ketones, butadienes, ketones, esters, acetals, carbonates and combinations thereof. In one embodiment, the thermoplastic polymer is at least one of a polystyrene, a polymethyl methacrylate and a polycarbonate. Examples of monomers to form the thermoplastic polymer may include but are not limited to methyls, ethylenes, propylenes, methyl methacrylates, methylpentenes, vinylidene, vinylidene chloride, etherimides, ethylenechlorinates, urethanes, ethylene vinyl alcohols, fluoroplastics, carbonates, acrylonitrile-butadiene-styrenes, etheretherketones, ionomers, butylenes, phenylene oxides, sulphones, ethersulphones, phenylene sulphides, elastomers, ethylene terephthalate, naphthalene terephthalate, ethylene naphthalene and combinations thereof. In various examples, the polymeric material for fabricating the pattern template includes but is not limited to PMMA (poly(methyl methacrylate)), PDMS (polydimethylsiloxane), photoresist, electron-beam resist and SUB. In one example, PMMA is used for fabricating the pattern template. In another example, PDMS prepolymer and curing agent (e.g. mixed in about a 10:1 volume ratio) are cast onto the mold, subjected to degassing for 30 minutes in vacuum and curing at 70° C. for 3 hours to fabricate the pattern template.

In various embodiments, the imprinting temperature may be from about 100° C. to about 200° C., from about 110° C. to about 190° C., from about 120° C. to about 180° C., from about 130° C. to about 170° C., from about 140° C. to about 160° C., or from about 150° C. to about 155° C. In one embodiment, the imprint temperature is about 140° C.

In various embodiments, the imprinting pressure may be from about 1 MPa to about 5 MPa, from about 1.5 MPa to about 4.5 MPa, from about 2 MPa to about 4 MPa, from about 2.5 MPa to about 3.5 MPa, or from about 3 MPa to about 3.5 MPa. In one embodiment, the imprint pressure is about 4 MPa.

In various embodiments, the imprinting duration may be from about 4 minutes to about 20 minutes, from about 6 minutes to about 18 minutes, from about 8 minutes to about 16 minutes, from about 10 minutes to about 14 minutes, from about 12 minutes to about 14 minutes, from about 22 minutes to about 30 minutes, from about 24 minutes to about 28 minutes, or from about 25 minutes to about 26 minutes. In one embodiment, the imprinting duration is about 5 minutes.

In one example, the plurality of wells is imprinted by hot embossing the substrate on a silicon mold using an imprinter at a temperature of about 140° C. and a pressure of about 4 MPa for a duration of about 30 minutes.

In various embodiments, the plurality of wells may be arranged in an array. In various embodiments, the plurality of wells comprises a defined shape when viewed from the top of the pattern template. The shape of the plurality of wells may be in the form of a sphere, hemisphere, ellipsoid, cylinder, cube, cuboid, cone, truncated cone, pyramid (e.g. triangular pyramid, square pyramid, polygonal pyramid, right pyramid, oblique pyramid etc.), truncated pyramid and the like. When viewed from above the pattern template, each well may have a cross-section which is substantially circular, elliptical or oval-shaped, triangle-shaped, square-shaped, trapezoidal-shaped, parallelogram-shaped, rectangular-shaped, polygon-shaped and the like. In various embodiments, depending on the geometry of each well, the longest dimension (i.e. one of height, length, width, or diameter) of each well may be from about 50 μm to about 1000 μm, from about 100 μm to about 950 μm, from about 150 μm to about 900 μm, from about 200 μm to about 850 μm, from about 250 μm to about 800 μm, from about 300 μm to about 750 μm, from about 350 μm to about 700 μm, from about 400 μm to about 650 μm, from about 450 μm to about 600 μm, or from about 500 μm to about 550 μm.

In various embodiments, the film which is deposited in the plurality of wells to form the plurality of chambers occupies from about 1% to about 10%, from 2% to about 9%, from 3% to about 8%, from 4% to about 7%, from 5% to about 6%, from about 10% to about 90%, from about 15% to about 85%, from about 20% to about 80%, from about 25% to about 75%, from about 30% to about 70%, from about 35% to about 65%, from about 40% to about 60%, from about 45% to about 55%, or from about 50% to about 55% by volume of each well, with the balance being occupied by the visual indicator configured to be stored within the chamber. In one example, the film which is deposited in the plurality of wells to form the plurality of chambers occupies from 1% to 20%, or from 1% to 10% by volume of each well, with the balance being occupied by the visual indicator configured to be stored within the chamber and/or air space.

In various embodiments, the method further comprises removing air bubbles from the plurality of wells in the pattern template. In one example, removing air bubbles from the plurality of wells in the pattern template comprises sonicating the pattern template in water for about 5 minutes. Sonicating may help to remove any air bubbles that may be trapped inside the plurality of wells in the pattern template. Removal of air bubbles may ensure that the film is deposited in a substantially uniform manner on the pattern template and prevent occurrence of discontinuity or breakage in the film that may occur if a bubble were present.

In various embodiments, the step of forming the film comprises depositing a material e.g. polymeric material on the pattern template to form a single layer e.g. a single layer of polymer. In various embodiments, the film comprising a single layer of polymer may be formed from a single polymeric material (e.g. poly(lactic) acid), or a composite/blend comprising two or more polymeric materials. In various embodiments, the step of forming the film comprises depositing two or more types of materials e.g. polymeric materials in an alternating manner to form alternating layers e.g. alternating complementary layers of polymers. In various embodiments, the alternating layers may be coupled via various types of interactions between the layers (and/or within layer pairs), which include but are not limited to hydrogen bonding, hydrophobic interactions, charge interactions etc. In one example, the film forming the plurality of chambers may be made from protein-polyphenol-clay layered composite material.

In various embodiments, the method further comprises dipping the pattern template in a first polyelectrolyte solution to form a first polymer layer as an initial anchoring layer on the pattern template. In various embodiments, the initial anchoring layer may be a positively charged layer. In various embodiments, the pattern template is treated with a cationic polymer solution. In one example, the pattern template is exposed for about 15 minutes to a branched poly(ethyleneimine) (PEI) solution with pH adjusted to about 5.0 using 1 M hydrochloric acid.

In various embodiments, the method further comprises dipping the pattern template in a second polyelectrolyte solution to facilitate adsorption of a second polymer layer onto the pattern template. In various embodiments, the method further comprises dipping the pattern template in a third polyelectrolyte solution to facilitate adsorption of a third polymer layer onto the pattern template. In various embodiments, the method further comprises forming alternating layers of a second and third polymer on the pattern template by dipping the pattern template in the second and third polyelectrolyte solutions in an alternating manner. For example, the step of forming the plurality of chambers may comprise alternately dipping the pattern template in a cationic polyelectrolyte solution and an anionic polyelectrolyte solution. The second polymer layer may be formed from a polyanionic material and the third polymer layer may be formed from a polycationic material. The step of alternately dipping the pattern template in the second and third polyelectrolyte solutions may be repeated until a film with the desired thickness or number of layers is formed. The thickness of each layer may be controlled by adjusting the pH or ionic strength of the polyelectrolyte solutions used for dipping the pattern template. As such, the thicknesses of the second polymer layer and third polymer layer may be substantially the same as, or different from each other.

Examples of polyanionic material include but are not limited to gum arabic, anionic polysaccharide, alginate, pectin, agar, carrageenan, polyacrylate, poly(4-styrenesulphonates), poly(vinylsulphonates), polyanetholesulphonates and combinations thereof. In one example, the anionic polyelectrolyte is poly(4-styrene sulfonate) (PSS).

Examples of polycationic material include but are not limited to gelatin (such as gelatin A or gelatin B), chitosan, whey proteins, albumin, beta-lactoglobulin, potato proteins, fava, legumin, soybean proteins, natural or synthetic polysaccharides such as chitosan, poly(allylammonium), poly(diallyldimethylammonium), poly(ethyleneimine) (PEI), polyl-lysine (PLL), poly(histidine), alginate, hyaluronic acid, heparin, heparan sulfate, chondroitin sulfate, dextran sulfate, poly(methacrylates), oxidized cellulose, carboxymethyl cellulose, polyaspartic acid, polyglutamic acid and combinations thereof. In one example, the cationic polyelectrolyte is poly (allylamine hydrochloride) (PAH).

In one example, the second polymer layer comprises PSS and the third polymer layer comprises PAH.

In various embodiments, adjacent polymer layers contacting each other have opposite electrostatic polarity. For example, if the first polymer layer is positively charged (e.g. formed from polycationic material), then the second polymer layer is negatively charged (e.g. formed from polyanionic material), and the third polymer layer is positively charged (e.g. formed from polycationic material). For example, if the first polymer layer is PEI, then the second polymer layer should be chosen from a group of polyanions, while the third polymer layer should be chosen from a group of polycations.

In various embodiments, the polyelectrolyte solution have an ionic strength of from about OM to about 5.5 M, from about 0.5 M to about 5 M, from about 1 M to about 4.5 M, from about 1.5 M to about 4 M, from about 2 M to about 3.5 M, from about 2 M to about 2.5 M, or from about 1 M to about 3 M.

In various embodiments, the polyelectrolyte solution comprises a polymer concentration from about 0.5 mg/ml to about 5 mg/ml, from about 1 mg/ml to about 4.5 mg/ml, from about 1.5 mg/ml to about 4 mg/ml, from about 2 mg/ml to about 3.5 mg/ml, or from about 2.5 mg/ml to about 3 mg/ml. In various examples, an upper limit for polymer concentration may be defined taking into account its solubility at a given temperature, viscosity of the solution (which also depends on the molecular weight), and practical considerations.

In various embodiments, the polyelectrolyte solution has a pH of from about 2.5 to about 8.5, from about 3 to about 8, from about 3.5 to about 7.5, from about 4 to about 7, from about 4.5 to about 6.5, from about 5 to about 6, or from about 5.5 to about 6. In various embodiments, the range of pH depends on the strength of polyacid/polybase. For relatively strong polyacids/polybases such as polystyrenesulfonate, the pH of the solution for coating may be less important as compared to relatively weak polyacids/polybases, as polyacrylic acid or polyallylamine, where the pH of the solution for coating may be more important. For quaternium ammonium salts, the pH of the solution for coating may not be as important.

In various embodiments, the pattern template is dipped into the polyelectrolyte solution for at least about 1 minute, at least about 3 minutes, at least about 5 minutes, at least about 10 minutes, at least about 15 minutes, at least about 20 minutes, at least about 25 minutes, at least about 30 minutes, at least about 35 minutes, at least about 40 minutes, at least about 45 minutes, or at least about 50 minutes. In various examples, the pattern template is dipped into the polyelectrolyte solution until the desired thickness of polymer layer is deposited on the pattern template.

In various embodiments, the method further comprising, subsequent to each of the said dipping step, washing the pattern template in a washing solution. In various embodiments, the step of washing the pattern template removes non-adsorbed macromolecules. In one example, the washing step comprises using water e.g. DI (deionized) water or salt solutions of a desired ionic strength to wash the pattern template. In various embodiments, the step of washing the pattern template lowers/decreases the concentration of free polyelectrolytes in the surrounding washing solution by e.g. at least 100 to 1000 times as compared to the concentration of the polyelectrolyte solution used for dipping the pattern template in the prior dipping step.

In various embodiments, the pattern template is washed for at least about seconds, at least about 1 minute, at least about 2 minutes, at least about 3 minutes, at least about 4 minutes, or at least about 5 minutes.

In various embodiments, the method further comprises applying/pressing a support layer to a film comprising the plurality of chambers to seal/cover the plurality of chambers to prevent and/or minimise leakage of the one or more visual indicators. The film may be a multilayered film comprising the first and second polymer layers.

In various embodiments, the method further comprises, prior to applying the film to the support layer, applying an adhesive layer between the film and the support layer. In various examples, the thickness and composition of an adhesive layer depend on factors such as the material of support, plurality of chambers e.g. microchambers and the dimensions of the microchambers. In various examples, the adhesive layer is adhesive to both film and support layer. In various examples, the adhesive layer does not fill into the plurality of chambers e.g. microchambers. In various examples, the adhesive layer may be chosen from a group of pressure-sensitive adhesives, or UV-curable adhesives. In one example, the adhesive layer is a PEM (polyelectrolyte multilayer) film made of poly(diallyldimethylammonium) and poly(4-styrenesulfonate), e.g. (PSS_PDADMA).

In various embodiments, the method further comprises removing the pattern template. The pattern template may be removed by dissolving the pattern template in an organic solvent or by mechanically peeling the pattern template. The step of dissolving the pattern template may comprise selecting an organic solvent that selectively dissolves the pattern template but does not dissolve the polymer(s) or component(s) making up the film, support layer and adhesive layer. The organic solvent may be an aromatic solvent such as toluene or benzene. The organic solvent may be an aliphatic solvent such as chloroform, methyl ethyl ketone, methyl isobutyl ketone, dimethyl sulphoxide (DMSO), N-methyl-2-pyrrolidone (NMP), dichloromethane (DCM) or tetrahydrofuran (THF). In an embodiment where the pattern template is PMMA, the organic solvent used is toluene. The step of dissolving the pattern template may further comprise removing the organic solvent. The organic solvent may be removed by drying e.g. evaporation of the organic solvent. Alternatively, the pattern template may be mechanically peeled out instead of dissolving in an organic solvent. In various examples, the peeled-out pattern substrate may be reusable.

In various embodiments, the method further comprises introducing/loading the plurality of chambers with one or more visual indicators. In various embodiments, the plurality of chambers is loaded/encapsulated with one or more visual indicators using a solvent-exchange method. In various embodiments, the one or more visual indicators comprises a dye compound dissolved in a solvent. In various examples, the dye is loaded by a solvent-exchange method by relying on solvents that do not evaporate visibly at room temperature, e.g. cooking oils, and which diffusion through membrane is hindered so that they are not washed away during the next washing step.

In various embodiments, the volume of visual indicator loaded into each chamber may be from about 0.5 nL to about 500 nL, from about 1 nL to about 450 nL, from about 1.5 nL to about 400 nL, from about 2 nL to about 450 nL, from about 3 nL to about 400 nL, from about 4 nL to about 350 nL, from about nL to about 300 nL, from about 10 nL to about 250 nL, from about 20 nL to about 200 nL, from about 30 nL to about 150 nL, from about 40 nL to about 100 nL, from about 50 nL to about 90 nL, from about 60 nL to about 80 nL, or from about 65 nL to about 70 nL.

In various embodiments, the method further comprises washing away excess one or more visual indicators using an organic solvent. In various examples, the organic solvent includes but is not limited to toluene.

In various embodiments, the method further comprises drying the device. In various examples, drying the device refers to evaporation of the organic solvent e.g. toluene from the device.

In various embodiments, the method of making the pressure sensing device may comprise applying a first film layer on a template to form an array of chambers; applying a second sealing layer to the array of chambers; removing the template from the first layer; and loading a dye solution into the chambers.

In various embodiments, the method of making the pressure sensing device may comprise applying a first film layer on a template to form an array of chambers; loading a dye solution into the chambers; applying a second sealing layer to the array of chambers; and removing the template from the first layer.

In various embodiments, there is provided a bandaging system comprising one or more pressure sensing devices as disclosed herein. In various embodiments, the bandaging system may be used in compression therapy for patients suffering from limb ulcers.

In various embodiments, the plurality of chambers e.g. microchambers containing/holding/storing one or more visual indicators are configured to release the one or more visual indicators when the pressure applied on the plurality of chambers exceeds the one or more pressure thresholds. In various embodiments, the collapse of the plurality of chambers e.g. microchambers loaded with dye-containing liquid, can be used to signal the pressure achieved under compression bandaging. For example, the microchambers can be designed to collapse when the compressive pressure achieved exceeds 20 mmHg under compression bandaging. In various embodiments, the pressure sensor relies on release of dye that is capable of penetrating through all layers of compression bandaging above the pressure sensor, thus signaling the actual pressure achieved. Typically, commercial compression systems consist of 2 or 4 layers of bandaging material/fabric.

In various embodiments, the bandaging system further comprises a primary/first bandaging layer for covering a site of interest. The site of interest may be a site of treatment such as a skin surface of a lower limb of a subject. The site of treatment may have one or more wounds e.g. ulcers formed on the skin surface of the subject. In one example, the subject may be a mammal which includes but is not limited to human, non-human primates, and the like. In various embodiments, the primary/first bandaging layer may function to protect the skin surface, prevent pain, prevent infection and/or contamination. In various embodiments, the one or more pressure sensing devices are positioned on the primary bandaging layer at various locations at the site of interest. The one or more pressure sensing devices may be placed at strategic locations where detection of applied pressure is desired. It will be appreciated that the one or more pressure sensing devices are placed in the vicinity of the one or more wounds and are not placed on the primary bandaging layer directly above the one or more wounds.

In various embodiments, the bandaging system further comprises one or more secondary bandaging layer for exerting pressure on the site of interest. In various embodiments, the one or more secondary bandaging layers are arranged to cover the primary bandaging layer and the one or more pressure sensing devices positioned on the primary bandaging layer. That is, when in use, the one or more pressure sensing devices are disposed between the primary bandaging layer and the one or more secondary bandaging layers.

In various embodiments, the one or more visual indicators are arranged to penetrate/wet the one or more secondary bandaging layers to produce/form one or more visual signals e.g. visually observable markings thereon when they are released from the plurality of chambers. In various embodiments, the one or more visual signals e.g. visually observable markings/stains on the one or more secondary bandaging layers indicate that the one or more pressure thresholds are exceeded.

In various embodiments, the one or more pressure sensing devices may be standalone devices which are not integrated into the fabric of the bandages. As such, the one or more pressure sensing devices may be removably attached to the primary bandaging layer or the one or more secondary bandaging layers.

In various embodiments, the one or more pressure sensing devices may be integrated into the bandage fabric. In various embodiments, the one or more pressure sensing devices may be disposable and configured for single use.

In various embodiments, there is provided a method of detecting applied pressure, the method comprising, positioning one or more pressure sensing devices as disclosed herein on a site of interest, applying pressure on the site of interest, and detecting one or more visually observable signals formed by the one or more visual indicators released from the plurality of chambers.

In various embodiments, the method further comprises, prior to positioning the one or more pressure sensing devices, covering the site of interest with a primary/first bandaging layer. The site of interest may be a site of treatment such as a skin surface of a lower limb of a subject where one or more ulcers are located. In various embodiments, the primary/first bandaging layer may function to protect the skin surface, prevent pain, prevent infection and/or contamination.

In various embodiments, the step of positioning the one or more pressure sensing devices comprises placing the one or more pressure sensing devices at various locations at the site of interest. The one or more pressure sensing devices may be placed at strategic locations where detection of applied pressure is desired. In various embodiments, the one or more pressure sensing devices are placed on the surface of the primary bandaging layer to avoid direct contact between the surface of the subject and the one or more pressure sensing devices.

In various embodiments, the method further comprises applying one or more secondary bandaging layers for exerting pressure on the surface of the subject. In various embodiments, the one or more secondary bandaging layers are arranged to cover the primary bandaging layer and the one or more pressure sensing devices positioned on the primary bandaging layer. In various embodiments, detection of the release of one or more visual indicators is through one or more visually observable signals formed on the one or more secondary bandaging layers e.g. surface of the one or more secondary bandaging layers.

In various embodiments, the step of detecting one or more visually observable signals comprises detecting a first marking when the pressure applied on the site of interest exceeds a first pressure threshold, and additionally detecting a second marking when the pressure applied on the site of interest exceeds a second pressure threshold. In various embodiments, the pressure applied on the site of interest is transmitted to the one or more pressure sensing devices placed at various locations at the site of interest. In various embodiments, the first marking is produced by the first visual indicator released from the first subset of chambers when the pressure exerted on the plurality of chambers exceeds a first pressure threshold. In various embodiments, the second marking is produced by the second visual indicator released from the second subset of chambers when the pressure exerted on the plurality of chambers exceeds a second pressure threshold.

In various embodiments, the step of applying pressure comprises increasing pressure on the site of interest until release of the first visual indicator is detected but before release of the second visual indicator is detected, indicating that the pressure applied on the site of interest exceeds the first pressure threshold but not the second pressure threshold. In various embodiments, the step of applying pressure optionally comprises decreasing pressure on the site of interest when release of the second visual indicator is detected, indicating that the pressure applied on the site of interest exceeds the second pressure threshold. In various embodiments, the pressure applied by the one or more secondary bandaging layers may be increased gradually. In various embodiments, the applied pressure may be increased such that only the first marking and not the second marking is formed. The formation of the first marking may provide an indication that the pressure applied on the site of interest is between the first pressure threshold and the second pressure threshold. In an application for compression bandaging, the first pressure threshold may be set to represent a minimum pressure threshold for informing a user that the pressure applied to the site of treatment is effective for compressive therapy.

In various embodiments, the step of applying pressure on the site of interest further comprises maintaining the pressure applied by the one or more secondary bandaging layers. In various embodiments, the applied pressure may be maintained such that the second marking is not formed. The formation of the second marking may provide an indication that the pressure applied on the site of interest is above the second pressure threshold. In an application for compression bandaging, the second pressure threshold may be set to represent a maximum pressure threshold for informing the user that the pressure applied to the site of treatment is unsafe. This may provide a warning signal to the user that the applied pressure is excessive and that the one or more secondary bandages may need to be reapplied with a safe and therapeutic level of pressure which is between the first and second pressure thresholds.

### BRIEF DESCRIPTION OF FIGURES

FIG. 1 is a schematic drawing of a pressure sensing device in an example embodiment.

FIG. 2 is a schematic drawing of a process/method of making/fabricating a pressure sensing device in an example embodiment.

FIG. 3A is a Scanning Electron Microscope (SEM) image of a pressure sensing device comprising multilayer films with arrays of sealed microchambers in an example embodiment. Scale bar=100 μm.

FIG. 3B is a magnified SEM image showing a microchamber of the pressure sensing device in the example embodiment. Scale bar=10 μm.

FIG. 4 is a fluorescent microscope image of microchambers filled with oil-soluble fluorescent dye in the example embodiment. Scale bar=300 μm.

FIG. 5A is a fluorescent microscope image of a prototype film sensor containing an array of microchambers filled with a fluorescent dye, prior to application of compressive pressure, in an example embodiment. Scale bar=300 μm.

FIG. 5B is a photograph of the prototype film sensor in the example embodiment.

FIG. 5C is a photograph of the prototype film sensors placed onto a non-compression bandage (upper arrow) and illuminated with blue light (bottom arrow) in the example embodiment.

FIG. 6A is a fluorescent microscope image of a compression bandage stained with fluorescent dye released from a prototype film sensor containing an array of microchambers filled with the fluorescent dye, after application of 22 mmHg compressive pressure, in an example embodiment. Scale bar=300 μm.

FIG. 6B is a photograph of the compression bandage in the example embodiment.

FIG. 6C is a photograph of the compression bandage with the prototype film sensor which is positioned under the compression bandage in the example embodiment upon illumination with blue light.

FIG. 7A is a photograph showing placement of disposable film sensors onto a first bandaging layer in an example embodiment.

FIG. 7B is a photograph showing application of compressive bandaging over the first bandaging layer in the example embodiment.

FIG. 7C is a photograph showing penetration of a dye from the film sensor through the compressive bandaging, signaling that a therapeutic level of compression (20 mmHg) is achieved in the example embodiment.

### DETAILED DESCRIPTION OF FIGURES

Example embodiments of the disclosure will be better understood and readily apparent to one of ordinary skill in the art from the following discussions and if applicable, in conjunction with the figures. It should be appreciated that other modifications related to structural, and optical changes may be made without deviating from the scope of the invention. Example embodiments are not necessarily mutually exclusive as some may be combined with one or more embodiments to form new exemplary embodiments.

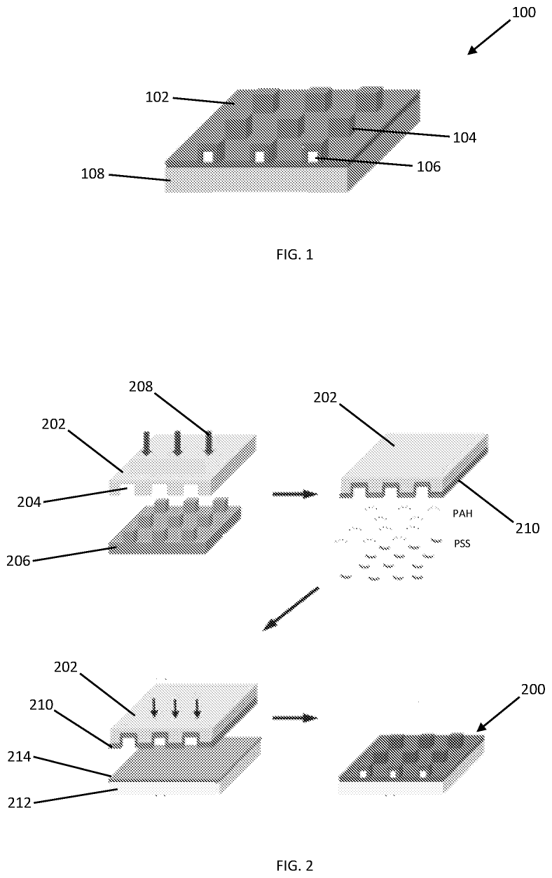

FIG. 1 is a schematic drawing of a pressure sensing device 100 in an example embodiment. The pressure sensing device/pressure sensor 100 comprises a relatively thin multilayer film containing an array of microchambers (i.e. micron-sized chambers) e.g. 104. Each microchamber 104 comprises walls which define an internal space 106 configured to be filled with a visual indicator e.g. a dye solution. The array of microchambers e.g. 104 are covered/sealed towards a support layer 108 e.g. a support layer made from polyethylene. Under compression, the microchambers e.g. 104 rupture upon reaching a critical pressure and release the dye.

In the example embodiment, the pressure sensing device 100 is in the form of a sheet e.g. strip and is configured to provide an indication e.g. visual signal once a specific pressure level is reached. For example, a first strip of the pressure sensing device 100 may be configured to release dye at an applied pressure of more than 20 mmHg. A second strip of the pressure sensing device 100 may be configured to release dye if the applied pressure is more than 40 mmHg. The geometry of the microchambers e.g. 104, thickness and composition of the multilayer film 102 are tuned to achieve the required critical pressures for dye release. For example, the microchambers designed to collapse at more than mmHg may be squarish protrusions/posts measuring about 150 μm by about 150 μm in length and width, with a height of about 100 μm and a film thickness of approximately 2.5 μm made from poly(styrene sulfonate)—poly(allylamine) multilayers. A substantially uniform pressure of 22 mmHg exerted on a sample pressure sensing device by a bandage results in the microchambers' collapse and release of a colour payload, which is observable by a naked eye.

The pressure sensing device 100 may be applied as a single-use-sensor to be integrated into compression bandages to effectively indicate 2 levels of compressive pressures at more than 20 mmHg and more than 40 mmHg. The pressure sensing device 100 may be used towards improving the efficiency of compressive therapy for chronic leg ulcers of venous or mixed aetiology. For example, the first strip may be used to indicate that the optimal therapeutic pressure is achieved to promote effective healing of an affected limb. The second strip may be used to indicate that the applied pressure is unsafe and may cause tissue necrosis, which means that the bandage must be reapplied.

FIG. 2 is a schematic drawing of a process/method of making/fabricating a pressure sensing device 200 in an example embodiment.

The process involves fabrication of a template (i.e. pattern template) 202 comprising an array of microwells e.g. 204 using a mold e.g. silicon mold 206. The pattern template 202 is made from a free-standing poly(methylmethacrylate) (PMMA) film/sheet. Each microwell 204 has dimensions of 150 μm by 150 μm by 100 μm (width: length: depth). The array of microwells e.g. 204 is fabricated on the surface of the PMMA film by hot embossing using an Obducat imprinter at 140° C. and 4 MPa (40 bar). As shown by the arrows e.g. 208, the pattern of the array of microwells e.g. 204 is imprinted onto the PMMA film, forming the pattern template 202.

The pattern template 202 which has been imprinted with the array of microwells e.g. 204 is then coated with polyelectrolyte multilayers (PEMs) 210 via Layer-by-Layer (LbL) assembly using a dip-coating robot machine (Riegler & Kirstein GmbH, Germany).

Prior to dip-coating, the pattern template 202 is sonicated in water for 5 minutes to remove air bubbles that may be trapped inside the microwells e.g. 204. The pattern template 202 is first exposed for 15 minutes to branched PEI (polyethylenimine) solution (with pH adjusted to 5.0 using 1 M HCl) in order to generate the first anchoring layer with high density of positive charges. Further alternating layers of poly(sodium 4-styrene sulfonate) (PSS) and poly(allylamine hydrochloride) (PAH) are introduced followed by three washings to remove all non-adsorbed macromolecules. Adsorption of all polyelectrolytes is performed from NaCl solutions of ionic strength 2M, polymer concentration 2 mg/mL, pH˜5.5, 30 min and 1 min for each adsorption and DI water washing step, respectively. The terminal layer is PSS.

Subsequently, the microchambers are sealed properly in order to effectively prevent leakage of loaded dyes from the pressure sensor/pressure sensing device 200. The sealing is achieved upon pressing of a support layer 212 e.g. polyethylene film pre-coated with an adhesive layer 214 e.g. approximately 0.5 μm thick adhesive multilayer against the pattern template 202 in a pressurized chamber at 1 MPa and 30° C. for 60 min, thereby forming films with sealed hollow microchambers on polyethylene support.

Finally, the pattern template 202 is dissolved in toluene followed by introduction of 0.1 mL of sunflower oil with dissolved fluorescent dye (3,4,9,10-tetra-(hectoxy-carbonyl)-perylene). After 12 hours, excess oil is washed out with toluene and the pressure sensor 200 is allowed to dry.

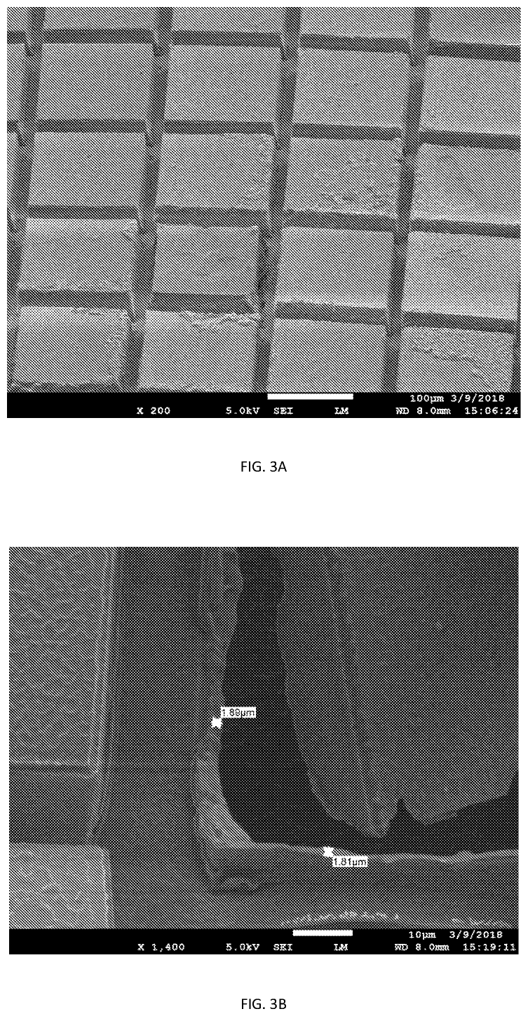

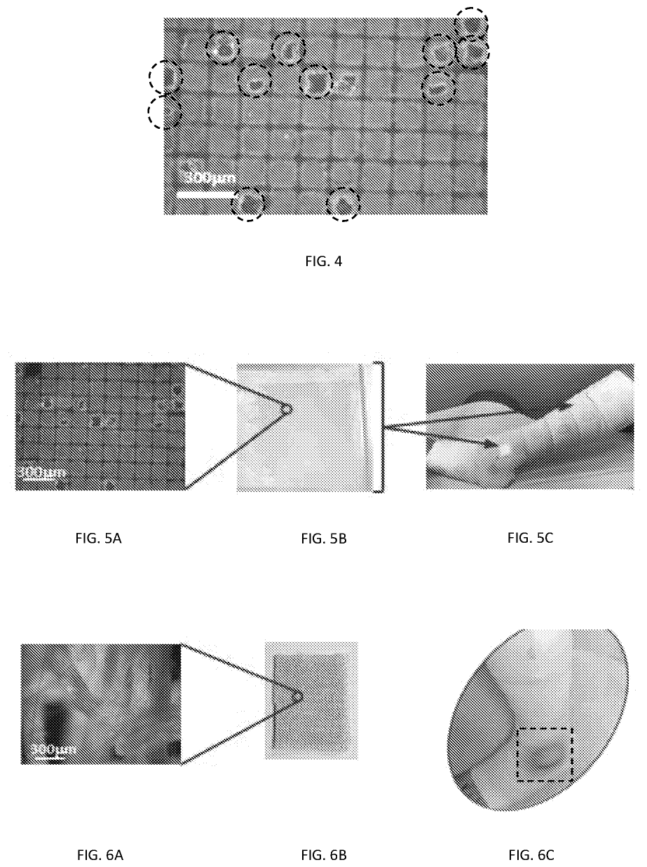

The pressure sensing device 200 that is fabricated in the example embodiment is then imaged using scanning electron and fluorescent microscopy to characterise the microchambers. FIG. 3A is a SEM image of the pressure sensing device 200 comprising multilayer films with arrays of sealed microchambers in the example embodiment. FIG. 3B is a magnified SEM image showing a microchamber of the pressure sensing device in the example embodiment. FIG. 4 is a fluorescent microscope image of microchambers filled with oil-soluble fluorescent dye in the example embodiment. The pressure sensing device/pressure sensor comprises an array of chambers measuring 150:150:100 μm (W:L:H) made of a 1.8-1.9 μm thick poly(styrene sulfonate) poly(allylamine) multilayer film. Each chamber is filled with a dye solution and sealed towards a polyethylene support layer. As shown in FIG. 4, microchambers loaded with oil-soluble dye emit fluorescence (i.e. lighted areas in the microscope image come from the fluorescent dye). The dotted circles demarcating darker areas in FIG. 4 are likely undissolved residues of the PMMA template. The microchambers are configured to rupture and release the dye when compression exceeds a critical threshold.

Evaluation of a pressure sensor at various pressure points is achieved by using an Instron 5569 test set up. Each sample (i.e. pressure sensor/film) is positioned in the center of a floating stage with a piece of equal-sized bandage placed on top of the sample. Uniform pressure is exerted on the sample with bandage from 2 mmHg onwards to 22 mmHg. An example of a prototype film sensor prior to application of compressive pressure is shown in FIG. 5B. A magnified view of a portion of the prototype film sensor under a fluorescent microscope, prior to application of compressive pressure, is shown in FIG. 5A. As shown in FIG. 5A, the prototype film sensor contains an array of microchambers filled with fluorescent dye (i.e. lighted areas in the microscope image come from the fluorescent dye). FIG. 5C shows intact prototype film sensors placed (i.e. covered) under compressive bandaging. The region where one of the film sensors is located is illuminated with a blue coloured light to excite the fluorescent dye, causing the dye to emit fluorescence. Fluorescent light emitted by the dye contained within the film sensor comes through the compressive bandaging material and becomes visible to the naked eye (see bottom arrow in FIG. 5C). In one sample pressure sensor/film, dye is released at an applied pressure of about 20 mmHg which is the optimal therapeutic pressure that promotes effective healing of an affected limb in compression therapy. The released dye penetrates through the bandaging and is observed on the outer surface by naked eye. FIG. 6B shows the bandaging material stained with a yellow dye released from the sample pressure sensor (note: as the images in the specification are reproduced in grayscale, the yellow dye stain may not be clearly visible). FIG. 6A is a magnified view of a portion of the bandaging material under a fluorescent microscope, showing the fibres of the bandaging material stained with the yellow dye. The sample pressure sensor contains 150×150×100 μm (L×W×H) microchambers made of about 1.8-1.9 μm thick PSS-PAH film, as shown in FIG. 3A and FIG. 3B.

The sample pressure sensor is then tested in a compression bandaging system by positioning on top of a first (non-compression) bandage layer. A second compression bandaging layer is then applied according to manufacturer's instructions. The dye is released when the applied pressure reaches therapeutic level of 20 mmHg at the ankle. The dye forms a mark/stain on the second compression bandaging layer.

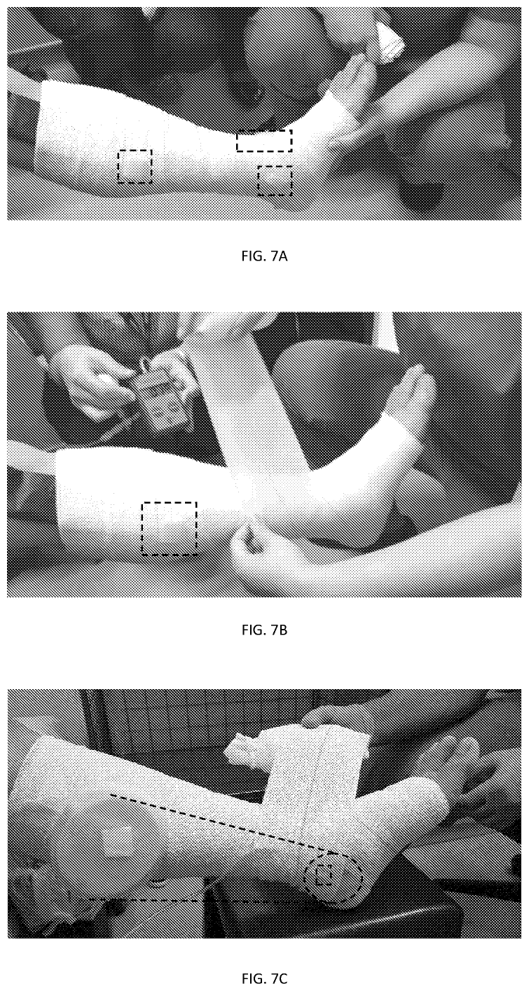

FIG. 7A to FIG. 7C shows an example of the pressure sensor as disclosed herein being used in a compression bandaging system. A first bandaging layer is applied onto an affected leg. Disposable film sensors are then placed onto the first bandaging layer at various locations. The locations of the film sensors are indicated by the dotted squares as shown in FIG. 7A. A second compressive bandaging layer is then applied over the first bandaging layer and covering the disposable film sensors positioned at the various locations. The dotted square in FIG. 7B shows a film sensor which has yet to be covered by the second compressive bandaging layer. Penetration of dye released from the film sensors through the compressive bandaging signals that a therapeutic level of compression (20 mmHg) is achieved in the example embodiment. As shown in FIG. 7C, the region where one of the film sensors is located is demarcated by a dotted circle. Yellow coloured dye released from ruptured chambers of the film sensor penetrates the compressive bandaging and forms a yellow coloured stain on the compressive bandaging (note: as the yellow coloured stain cannot be clearly visualized from the grayscale photograph, the location of the stain is indicated by the area demarcated by a dotted rectangle). FIG. 7C also shows a magnified image of the film sensor in the shape of a square without the compressive bandaging. The film sensor is stained yellow from the dye released from the ruptured chambers, after a compressive pressure of about 20 mmHg is applied.

### Applications

Embodiments of the disclosure provided herein provide a pressure sensing device and a method of making a pressure sensing device. In various embodiments, the pressure sensing device is capable of providing an indication of the amount of pressure being exerted thereon.

In various embodiments, the pressure sensing device is simple and easy to use by providing visual signals/indications (e.g. colour markings) which can be observed using the naked eye. Example embodiments of the pressure sensing device are relatively cheap to fabricate and are configured for disposable single-use.

In various embodiments, the pressure sensing device may be used in conjunction with compressive therapy for chronic leg ulcers of mixed aetiology. Example embodiments of the pressure sensing device provide a thin film with an array of microchambers loaded with a dye. The array of microchambers collapses and releases the dye when a therapeutic level of compression is delivered to the affected limb. The released dye penetrates through multiple layers of bandaging, forming one or more markings on the bandage which helps to guide clinicians, nurses and patients.

Advantageously, the pressure sensing device may be configured to be relatively thin and substantially flexible, such that its presence may not be substantially felt when placed under bandaging. The pressure sensing device is capable of working with existing compression bandaging systems and does not need to be integrated into the fabric of a bandage. Placement of the pressure sensing device between a primary bandaging layer and a secondary compressive bandaging layer does not affect uniformity of delivered compression. The pressure sensing device also does not require additional components (e.g. electronic components such as transducers) to function.

Even more advantageously, the pressure sensing device may effectively provide an indication of therapeutic or dangerously high compressive pressure achieved under the bandaging due to the ability to release dye when critical pressure is applied. For example, the pressure sensing device may guide a user e.g. healthcare professionals, untrained nurses by providing a visual indication when therapeutic compression is achieved, as well as to warn/alarm the user when/if compression reaches dangerously high level. This may help to overcome fear of causing harm and ensure effective administration of compression therapy.

Even more advantageously, the pressure sensing device may benefit patients undergoing compression therapy by helping patients avoid lengthy ineffective therapies and complications as serious as loss of limb. The pressure sensing device may also help to enhance healing rates of compression therapy, delay the onset of disability and improve quality of life. The pressure sensing device may also alarm patients in out-of-clinic settings, at any point of time after the application of compression bandaging, when/if the bandage or sub-bandage pressure reaches dangerously high levels.

It will be appreciated by a person skilled in the art that other variations and/or modifications may be made to the embodiments disclosed herein without departing from the spirit or scope of the disclosure as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

1. A pressure sensing device comprising,
a plurality of chambers for storing one or more visual indicators,
wherein the plurality of chambers are configured to release the one or more visual indicators when a pressure exerted on the plurality of chambers exceeds one or more pressure thresholds.

2. The device according to claim 1, wherein the plurality of chambers comprise,
a first subset of chambers configured to be collapsible to release a first visual indicator contained therein, when the pressure exerted on the plurality of chambers exceeds a first pressure threshold,
a second subset of chambers configured to be collapsible to release a second visual indicator contained therein, when the pressure exerted on the plurality of chambers exceeds a second pressure threshold,
wherein the second pressure threshold is higher than the first pressure threshold.

3. The device according to claim 2, further comprising,
a first visual indicator stored within the first subset of chambers; and
a second visual indicator stored within the second subset of chambers,
wherein the release of only the first visual indicator indicates that the pressure exerted on the plurality of chambers is between the first and second pressure thresholds, and
wherein the release of both the first and second visual indicators indicates that the pressure exerted on the plurality of chambers is above the second pressure threshold.

4. The device according to claim 2 or 3,
wherein the first pressure threshold is set at a level between 15 mmHg to mmHg, and the second pressure threshold is set at a level between 25 mmHg to 45 mmHg.

5. The device according to any one of claims 1 to 4, wherein the second pressure threshold is at least 10 mmHg higher than the first pressure threshold.

6. The device according to any one of claims 1 to 5, wherein the pressure exerted on the plurality of chambers is a compressive pressure.

7. The device according to any one of claims 1 to 6, wherein the plurality of chambers comprise walls having a thickness of from 1 μm to 10 μm.

8. The device according to any one of claims 1 to 7, wherein the plurality of chambers is formed by a film comprising a single layer of polymer.

9. The device according to any one of claims 1 to 7, wherein the plurality of chambers is formed by a film comprising alternating complementary layers of polymers,
wherein at least one alternating layer comprises polyanionic material selected from the group consisting of gum arabic, anionic polysaccharide, alginate, pectin, agar, carrageenan, polyacrylate, poly(4-styrenesulphonates), poly(vinylsulphonates), polyanetholesulphonates and combinations thereof; and
wherein at least another alternating layer comprises polycationic material selected from the group consisting of gelatin, chitosan, whey proteins, albumin, beta-lactoglobulin, potato proteins, fava, legumin, soybean proteins, natural or synthetic polysaccharides, chitosan, poly(allylammonium), poly(diallyldimethylammonium), poly(ethyleneimine) (PEI), polyl-lysine (PLL), poly(histidine), heparin, heparan sulfate, chondroitin sulfate, dextran sulfate, oxidized cellulose, polyaspartic acid, polyglutamic acid and combinations thereof.

10. The device according to claim 9, wherein the film comprises at least layers of polymers.

11. The device according to any one of claims 1 to 10, wherein the plurality of chambers is sealed by a support layer to prevent and/or minimise leakage of the one or more visual indicators from said chambers.

12. A method of making a pressure sensing device, the method comprising,
forming a film comprising a plurality of chambers for storing one or more visual indicators,
wherein the plurality of chambers are configured to release the one or more visual indicators when a pressure exerted on the plurality of chambers exceeds one or more pressure thresholds.

13. The method according to claim 12, further comprising, prior to forming the film, fabricating a pattern template comprising a plurality of wells, wherein said plurality of wells is a template for the plurality of chambers.

14. The method according to claim 12 or 13, wherein the step of forming the film comprises depositing a polymeric material on the pattern template to form a single layer of polymer, or depositing two or more polymeric materials in an alternating manner to form alternating layers of complementary polymers.

15. The method according to any one of claims 12 to 14, wherein the step of forming the film comprises forming a plurality of chambers with walls having a thickness of from 1 μm to 10 μm.

16. The method according to any one of claims 12 to 15, further comprising applying a support layer to the film to prevent and/or minimise leakage of the one or more visual indicators from the plurality of chambers.

17. The method according to any one of claims 12 to 16, further comprising loading one or more visual indicators into the plurality of chambers using an ink-jet printer or a solvent exchange technique.

18. A bandaging system comprising,
one or more pressure sensing devices as defined in any one of claims 1 to 11.

19. The bandaging system according to claim 18, further comprising a primary bandaging layer for covering a site of interest, wherein the one or more pressure sensing devices are arranged to be positioned on the primary bandaging layer.

20. The bandaging system according to claim 19, further comprising one or more secondary bandaging layers for exerting pressure on the site of interest, wherein the one or more secondary bandaging layers are arranged to cover the one or more pressure sensing devices positioned on the primary bandaging layer.

21. The bandaging system according to claim 20, wherein the one or more visual indicators are arranged to penetrate the one or more secondary bandaging layers to form one or more visually observable markings thereon when they are released from the plurality of chambers.

22. A method of detecting applied pressure, the method comprising,
positioning one or more pressure sensing devices as defined in any one of claims 1 to 11 on a site of interest,
applying pressure on the site of interest, and
detecting one or more visually observable signals formed by the one or more visual indicators released from the plurality of chambers.

23. The method according to claim 22, wherein the step of applying pressure comprises,
increasing pressure on the site of interest until release of a first visual indicator is detected but before release of a second visual indicator is detected, indicating that the pressure applied on the site of interest exceeds a first pressure threshold but not a second pressure threshold.

24. The method according to claim 22 or 23, further comprising, prior to positioning the one or more pressure sensing devices, covering the site of interest with a primary bandaging layer.

25. The method according to claim 24, further comprising applying one or more secondary bandaging layers for exerting pressure on the site of interest, wherein the one or more secondary bandaging layers are arranged to cover the one or more pressure sensing devices positioned on the primary bandaging layer.

26. The method according to claim 25, wherein detection of the release of one or more visual indicators is through one or more visually observable signals formed on the one or more secondary bandaging layers.
